(19) United States
(12) Patent Application Publication
(10) Pub. No.: US 2004/0058946 A1
(43) Pub. Date: Mar. 25, 2004

(54) Abuse-resistant prodrugs of oxycodone and other pharmaceuticals

(76) Inventors: Stephen L. Buchwald, Newton, MA (US); Timothy M. Swager, Newton, MA (US); Roman V. Rariy, Allston, MA (US)

Correspondence Address: FOLEY HOAG, LLP, PATENT GROUP, WORLD TRADE CENTER WEST, 155 SEAPORT BLVD, BOSTON, MA, 02110, US

(21) Appl. No.: 10/613,742

(22) Filed: Jul. 3, 2003

Related U.S. Application Data
(60) Provisional application No. 60/393,876, filed on Jul. 5, 2002.
(60) Provisional application No. 60/463,514, filed on Apr. 15, 2003.

(51) Int. Cl. A61K 31/517; A61K 31/485

(52) U.S. Cl. 514/282; 546/45; 514/270; 514/252.16; 544/284; 544/302

(57) ABSTRACT

One aspect of the present invention relates to compositions and methods for discouraging improper use of habit-forming and addictive drugs, such as oxycodone. In a preferred embodiment, a habit-forming or addictive drug is chemically modified to block its physiological activity until the drug is transformed to a physiologically active form in the mammalian gastrointestinal tract.

### RELATED APPLICATIONS

This application claims the benefit of priority to U.S. Provisional Patent Application serial No. 60/393,876, filed Jul. 5, 2002; and U.S. Provisional Patent Application serial No. 60/463,514, filed Apr. 15, 2003.

### BACKGROUND OF THE INVENTION

Oxycodone, morphine, and many other drugs are successful and therapeutically useful medications, e.g., as pain killers, when administered orally. Unfortunately, they also pose a severe threat for willful abuse when injected or snorted. Oxycodone is a controlled substance in Schedule II of the Controlled Substances Act (CSA), which is administered by the Drug Enforcement Administration (DEA). Schedule II provides the maximum amount of control possible under the CSA for approved drug products.

The FDA recently strengthened the warnings and precautions sections in the labeling of OxyContin® (oxycodone HCl controlled-release) Tablets, a narcotic drug approved for the treatment of moderate to severe pain, because of continuing reports of abuse and diversion. OxyContin® contains oxycodone HCl, an opioid agonist with an addiction potential similar to that of morphine. Opioid agonists are substances believed to act by attaching to specific proteins called opioid receptors, which are found in the brain, spinal cord, and gastrointestinal tract. When these drugs attach to certain opioid receptors in the brain and spinal cord they can effectively block the transmission of pain messages to the brain.

There have been numerous reports of Oxycodone diversion and abuse in several states. Some of these reported cases have been associated with serious consequences including death. Oxycodone, like morphine, has a high potential for abuse. It is supplied in a controlled-release dosage form and is intended to provide up to 12 hours of relief from moderate to severe pain. The tablet must be taken whole and only by mouth. When the tablet is chewed, crushed and/or its contents are injected intravenously or snorted into the nostrils, the controlled release mechanism is defeated and a potentially lethal dose of oxycodone is released immediately.

Although abuse, misuse, and diversion are potential problems for all opioids, including oxycodone, opioids are a very important part of the medical armamentarium for the management of pain when used appropriately under the careful supervision of a physician.

Currently available formulations for such drugs allow oral administrations but do not preclude chewing, injections and snorting. The problems with abuse are significant and longstanding, and repeated efforts to design new abuse-resistant formulations have been largely unsuccessful. In designing formulations of this sort, one should keep in mind that deliberate drug abusers and especially dealers are frequently quite sophisticated experimentally and capable of routinely performing such chemical operations as various aqueous and organic solvent extractions, neutralizations, etc. This reality makes simple anti-abuse approaches, e.g., including a solid acid into a formulation (which would be an abuse deterrent by causing burning if injected or snorted) or using capsules with shells insoluble in cold water, ineffective.

Therefore there exists a need to provide drug formulations that promote proper administration and usage of drugs, and are resistant to drug abuse.

### SUMMARY OF THE INVENTION

Several methods and compositions for encouraging proper administration and reducing the likelihood of abuse of drugs such as, for example, oxycodone, have been developed. The technology is useful for a number of other drugs where oral delivery is desired, and there is the potential for abuse or poor patient compliance if administered by any other route.

In part, the present invention relates to a compound comprising a drug bonded to an organic chain wherein cleavage between the drug and the organic chain under in vitro conditions is hindered, but cleavage under the in vivo conditions of the gastrointestinal tract where drug delivery is desired occurs at a faster rate. One, non-limiting way these compounds may be created is by forming an ester with a short organic chain (3-4 atoms) that has, at its distal end, an ester or amide linkage to an R group. Drug release may occur by a two-step mechanism following oral delivery to the gastrointestinal tract; the first step is the enzymatic cleavage of the distal ester or amide. In the second step, the hydroxy or amino group formed in the first step may participate in an intramolecular nucleophilic substitution reaction, cleaving the ester closest to the drug molecule, thereby releasing the drug. The nature of the R group may be varied to make the linkage more chemically robust, thereby preventing or minimizing liberation of active material by simple chemical methods such as acid and base treatment.

In part, the present invention relates to a pharmaceutical composition comprising a compound of the present invention and a pharmaceutically acceptable excipient.

In part, the present invention relates to a method of treating a mammal for pain relief comprising administering to the mammal a compound of the present invention wherein the compound comprises a pain relief drug chemically modified as described in the previous paragraph. In a further embodiment, the drug is oxycodone. In a further embodiment, the mammal is a primate, equine, canine, or feline. In a further embodiment, the mammal is a human.

In part, the present invention relates to a method for making a drug more abuse resistant comprising bonding an organic radical to the drug via a nucleophile on the drug, wherein the organic radical comprises at one end a chemical group susceptible to enzymatic cleavage and substantially non-susceptible to non-enzymatic cleavage, such that enzymatic cleavage liberates a nucleophile which may intramolecularly react to liberate the active form of the drug. In a further embodiment, the drug is a pain relief drug. In a further embodiment the drug is oxycodone.

In part, the present invention also provides for kits containing at least one dose of a subject composition, and often many doses, and other materials for a treatment regimen. For example, in one embodiment, a kit of the present invention contains sufficient subject composition for from five to thirty days and optionally equipment and supplies necessary to measure one or more indices relevant to the treatment regiment. In another embodiment, kits of the present invention contain all the materials and supplies, including subject compositions, for carrying out any methods of the present invention. In still another embodiment, kits of the present invention, as described above, additionally include instructions for the use and administration of the subject compositions.

In certain embodiments, the subject compounds may be formulated as a tablet, pill capsule or other appropriate ingestible formulation, to provide a therapeutic dose in 10 tablets or fewer. In another example, a therapeutic dose is provided in 50, 40, 30, 20, 15, 10, 5 or 3 tablets.

In certain embodiments, the present invention provides pain relief compositions, and methods of using the same, for the reduction and abatement of at least one painful disorder or condition based on a therapeutic regimen. In certain aspects, the present invention contemplates monitoring such disorder or condition as part of any therapeutic regimen, which may be administered over the short-term and/or long-term. These aspects of the invention may be particularly helpful in preventive care regimes.

In another aspect of the present invention, the pain relief compounds or compositions of the present invention may be used in the manufacture of a medicament to treat a pain related condition or disease. In certain embodiments, the present invention is directed to a method for formulating compounds of the present invention in a pharmaceutically acceptable carrier or excipient.

These embodiments of the present invention, other embodiments, and their features and characteristics, will be apparent from the description, drawings and claims that follow.

### BRIEF DESCRIPTION OF THE DRAWINGS

FIG. 1 depicts a schematic of oxycodone chemically modified to form an enzymatically activated prodrug. The arrow indicates the internal functional group that is too hindered to be cleaved either enzymatically or by ordinary chemical means. The distal group is substantially non-cleavable by ordinary chemical means and requires enzymatic methods.

FIG. 2 depicts a schematic of modified oxycodone demonstrating potential variations in the prodrug architecture, wherein W may be an organic chain of 3, 4 or 5 atoms that may bear substituents or be interrupted by non-carbon atoms including O, S, N, Si, or P; Y may be NH, S, or O; and R may be any organic group, including the carboxyl terminus of an N-acyl amino acid or the carboxy terminus of an oligopeptide.

### DETAILED DESCRIPTION OF THE INVENTION

Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

The term “ED₅₀” means the dose of a drug which produces 50% of its maximum response or effect. Alternatively, the dose which produces a pre-determined response in 50% of test subjects or preparations.

The term “LD₅₀” means the dose of a drug which is lethal in 50% of test subjects.

The term “therapeutic index” refers to the therapeutic index of a drug defined as LD₅₀/ED₅₀.

The term “structure-activity relationship (SAR)” refers to the way in which altering the molecular structure of drugs alters their interaction with a receptor, enzyme, etc.

The term “agonist” refers to a compound that mimics the action of natural transmitter or, when the natural transmitter is not known, causes changes at the receptor complex in the absence of other receptor ligands.

The term “antagonist” refers to a compound that binds to a receptor site, but does not cause any physiological changes unless another receptor ligand is present.

The term “inverse agonist” refers to a compound that binds to a constitutively active receptor site and reduces its physiological function.

The term “competitive antagonist” refers to a compound that binds to a receptor site; its effects can be overcome by increased concentration of the agonist.

The term “partial agonist” refers to a compound that binds to a receptor site but does not produce the maximal effect regardless of its concentration.

The term “ligand” refers to a compound that binds at the receptor site.

The terms “active agent”, “pharmacologically active agent” and “drug” are used interchangeably herein to refer to a chemical compound that induces a desired pharmacological, physiological effect. The terms also encompass pharmaceutically acceptable, pharmacologically active derivatives of those active agents specifically mentioned herein, including, but not limited to, salts, esters, amides, prodrugs, active metabolites, analogs, and the like. When the terms “active agent”, “pharmacologically active agent” and “drug” are used, or when a particular drug, such as oxycodone, is identified, it is to be understood as including the active agent per se as well as pharmaceutically acceptable, pharmacologically active salts, esters, amides, prodrugs, metabolites, analogs, etc.

The term “pharmaceutically acceptable salts” refers to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 20th ed., Lippincott Williams & Wilkins, Baltimore, Md., 2000, p. 704, the disclosure of which is hereby incorporated by reference.

The term “heteroatom” as used herein means an atom of any element other than carbon or hydrogen. Preferred heteroatoms are boron, nitrogen, oxygen, phosphorus, sulfur and selenium.

The term “alkyl” refers to the radical of saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has 30 or fewer carbon atoms in its backbone (e.g., C₁-C₃₀ for straight chain, C₃-C₃₀ for branched chain), and more preferably 20 or fewer. Likewise, cycloalkyls have from 3-10 carbon atoms in their ring structure, and more preferably have 5, 6 or 7 carbons in the ring structure.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths. Preferred alkyl groups are lower alkyls. In preferred embodiments, a substituent designated herein as alkyl is a lower alkyl.

The term “aralkyl”, as used herein, refers to an alkyl group substituted with an aryl group (e.g., an aromatic or heteroaromatic group).

The terms “alkenyl” and “alkynyl” refer to unsaturated aliphatic groups analogous in length and possible substitution to the alkyls described above, but that contain at least one double or triple bond respectively.

The term “aryl” as used herein includes 5-, 6- and 7-membered single-ring aromatic groups that may include from zero to four heteroatoms, for example, benzene, naphthalene, anthracene, pyrene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics.” The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

The terms ortho, meta and para apply to 1,2-, 1,3- and 1,4-disubstituted benzenes, respectively. For example, the names 1,2-dimethylbenzene and ortho-dimethylbenzene are synonymous.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The terms “polycyclyl” or “polycyclic group” refer to two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, alkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “carbocycle”, as used herein, refers to an aromatic or non-aromatic ring in which each atom of the ring is carbon.

As used herein, the term “nitro” means —NO₂; the term “halogen” designates —F, —Cl, —Br or —I; the term “sulfhydryl” means —SH; the term “hydroxyl” means —OH; and the term “sulfonyl” means —SO₂.

The terms “amine” and “amino” are art-recognized and refer to both unsubstituted and substituted amines, e.g., a moiety that can be represented by the general formula:

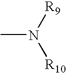

wherein R₉, R₁₀ and R′₁₀ each independently represent a hydrogen, an alkyl, an alkenyl, —(CH₂)ₘ—R₈, or R₉ and R₁₀ taken together with the N atom to which they are attached complete a heterocycle having from 4 to 8 atoms in the ring structure; R₈ represents an aryl, a cycloalkyl, a cycloalkenyl, a heterocycle or a polycycle; and m is zero or an integer in the range of 1 to 8. In certain embodiments, only one of R₉ or R₁₀ may be a carbonyl, e.g., R₉, R₁₀ and the nitrogen together do not form an imide. In other embodiments, R₉ and R₁₀ (and optionally R′₁₀) each independently represent a hydrogen, an alkyl, an alkenyl, or —(CH₂)ₘ—R₈. Thus, the term “alkylamine” as used herein means an amine group, as defined above, having a substituted or unsubstituted alkyl attached thereto, i.e., at least one of R₉ and R₁₀ is an alkyl group.

The term “acylamino” is art-recognized and refers to a moiety that can be represented by the general formula:

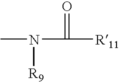

wherein R₉ is as defined above, and R′₁₁ represents a hydrogen, an alkyl, an alkenyl or —(CH₂)ₘ—R₈, where m and R₈ are as defined above.

The term “amido” is art recognized as an amino-substituted carbonyl and includes a moiety that can be represented by the general formula:

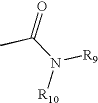

wherein R₉, R₁₀ are as defined above. Preferred embodiments of the amide will not include imides which may be unstable.

The term “alkylthio” refers to an alkyl group, as defined above, having a sulfur radical attached thereto. In preferred embodiments, the “alkylthio” moiety is represented by one of —S-alkyl, —S-alkenyl, —S-alkynyl, and —S—(CH₂)ₘ—R₈, wherein m and R₈ are defined above. Representative alkylthio groups include methylthio, ethyl thio, and the like.

The term “carbonyl” is art recognized and includes such moieties as can be represented by the general formula:

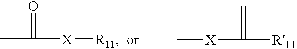

wherein X is a bond or represents an oxygen or a sulfur, and R₁₁ represents a hydrogen, an alkyl, an alkenyl, —(CH₂)ₘ—R₈ or a pharmaceutically acceptable salt, R′₁₁ represents a hydrogen, an alkyl, an alkenyl or —(CH₂)ₘ—R₈, where m and R₈ are as defined above. Where X is an oxygen and R₁₁ or R′₁₁ is not hydrogen, the formula represents an “ester”. Where X is an oxygen, and R₁₁ is as defined above, the moiety is referred to herein as a carboxyl group, and particularly when R₁₁ is a hydrogen, the formula represents a “carboxylic acid”. Where X is an oxygen, and R′₁₁ is hydrogen, the formula represents a “formate”. In general, where the oxygen atom of the above formula is replaced by sulfur, the formula represents a “thiolcarbonyl” group. Where X is a sulfur and R₁₁ or R′₁₁ is not hydrogen, the formula represents a “thiolester.” Where X is a sulfur and R₁₁ is hydrogen, the formula represents a “thiolcarboxylic acid.” Where X is a sulfur and R₁₁′ is hydrogen, the formula represents a “thiolformate.” On the other hand, where X is a bond, and R₁₁ is not hydrogen, the above formula represents a “ketone” group. Where X is a bond, and R₁₁ is hydrogen, the above formula represents an “aldehyde” group.

The terms “alkoxyl” or “alkoxy” as used herein refers to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —O—(CH₂)ₘ—R₈, where m and R₈ are described above.

The term “sulfonate” is art recognized and includes a moiety that can be represented by the general formula:

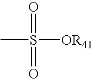

in which R₄₁ is an electron pair, hydrogen, alkyl, cycloalkyl, or aryl.

The terms triflyl, tosyl, mesyl, and nonaflyl are art-recognized and refer to trifluoromethanesulfonyl, p-toluenesulfonyl, methanesulfonyl, and nonafluorobutanesulfonyl groups, respectively. The terms triflate, tosylate, mesylate, and nonaflate are art-recognized and refer to trifluoromethanesulfonate ester, p-toluenesulfonate ester, methanesulfonate ester, and nonafluorobutanesulfonate ester functional groups and molecules that contain said groups, respectively.

The abbreviations Me, Et, Ph, Tf, Nf, Ts, and Ms represent methyl, ethyl, phenyl, trifluoromethanesulfonyl, nonafluorobutanesulfonyl, p-toluenesulfonyl and methanesulfonyl, respectively. A more comprehensive list of the abbreviations utilized by organic chemists of ordinary skill in the art appears in the first issue of each volume of the Journal of Organic Chemistry; this list is typically presented in a table entitled Standard List of Abbreviations. The abbreviations contained in said list, and all abbreviations utilized by organic chemists of ordinary skill in the art are hereby incorporated by reference.

The term “sulfate” is art recognized and includes a moiety that can be represented by the general formula:

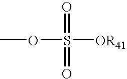

in which R₄₁ is as defined above.

The term “sulfonamido” is art recognized and includes a moiety that can be represented by the general formula:

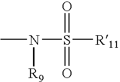

in which R₉ and R′₁₁ are as defined above.

The term “sulfamoyl” is art-recognized and includes a moiety that can be represented by the general formula:

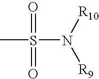

in which R₉ and R₁₀ are as defined above.

The term “sulfonyl”, as used herein, refers to a moiety that can be represented by the general formula:

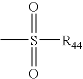

in which R₄₄ is selected from the group consisting of hydrogen, alkyl, alkenyl, alkynyl, cycloalkyl, heterocyclyl, aryl, or heteroaryl.

The term “sulfoxido” as used herein, refers to a moiety that can be represented by the general formula:

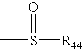

in which R₄₄ is selected from the group consisting of hydrogen, alkyl, alkenyl, alkynyl, cycloalkyl, heterocyclyl, aralkyl, or aryl.

A “phosphoryl” can in general be represented by the formula:

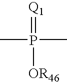

wherein Q₁ represented S or O, and R₄₆ represents hydrogen, a lower alkyl or an aryl. When used to substitute, e.g., an alkyl, the phosphoryl group of the phosphorylalkyl can be represented by the general formula:

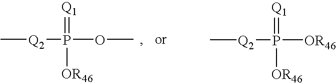

wherein Q₁ represented S or O, and each R₄₆ independently represents hydrogen, a lower alkyl or an aryl, Q₂ represents O, S or N. When Q₁ is an S, the phosphoryl moiety is a “phosphorothioate”.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g. alkyl, m, n, etc., when it occurs more than once in any structure, is intended to be independent of its definition elsewhere in the same structure.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. This invention is not intended to be limited in any manner by the permissible substituents of organic compounds.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

Certain compounds of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Contemplated equivalents of the compounds described above include compounds which otherwise correspond thereto, and which have the same general properties thereof, wherein one or more simple variations of substituents are made which do not adversely affect the efficacy of the compound. In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

For purposes of this invention, the chemical elements are identified in accordance with the Periodic Table of the Elements, CAS version, Handbook of Chemistry and Physics, 67th Ed., 1986-87, inside cover.

Compounds of the Present Invention

In part, the present invention relates to compounds of formula I:

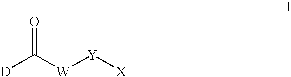

wherein:

D is a drug radical;

W is an organic chain comprising 3-5 carbon atoms that are substituted or unsubstituted and optionally comprises 3-5 heavy atoms selected from the group consisting of O, S, N, Si, and P;

Y is NH, S, or O; and

X is —CO-alkyl, —CO-aryl, —CO-aralkyl, —CO-heteroaryl, —CO-heteroaralkyl, —CO₂-alkyl, —CO₂-aryl, —CO-NHalkyl, —CO-NHaryl, the carboxy terminus of an N-acyl amino acid or the carboxy terminus of an oligopeptide.

In a further embodiment, the present invention relates to a compound of formula I and the attendant definitions, wherein D is a radical of alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, tramadol, allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, or zolazepam hydrochloride.

In a further embodiment, the present invention relates to a compound of formula I and the attendant definitions, wherein D is a radical of oxycodone.

In a further embodiment, the present invention relates to a compound of formula I and the attendant definitions, wherein W is —CH₂CH₂CH₂— or —CH₂CH₂CH₂CH₂—.

In a further embodiment, the present invention relates to a compound of formula I and the attendant definitions, wherein Y is NH.

In a further embodiment, the present invention relates to a compound of formula I and the attendant definitions, wherein X is —CO-alkyl or the carboxy terminus of an N-acyl amino acid or the carboxy terminus of an oligopeptide.

In a further embodiment, the present invention relates to a compound of formula I and the attendant definitions, wherein D is a radical of oxycodone and W is —CH₂CH₂CH₂— or CH₂CH₂CH₂CH₂—.

In a further embodiment, the present invention relates to a compound of formula I and the attendant definitions, wherein D is a radical of oxycodone and Y is NH.

In a further embodiment, the present invention relates to a compound of formula I and the attendant definitions, wherein D is a radical of oxycodone and X is —CO-alkyl or the carboxy terminus of an N-acyl amino acid or the carboxy terminus of an oligopeptide.

In a further embodiment, the present invention relates to a compound of formula I and the attendant definitions, wherein W is —CH₂CH₂CH₂— or —CH₂CH₂CH₂CH₂—; and Y is NH.

In a further embodiment, the present invention relates to a compound of formula I and the attendant definitions, wherein W is —CH₂CH₂CH₂— or —CH₂CH₂CH₂CH₂—; and X is —CO-alkyl or the carboxy terminus of an N-acyl amino acid or the carboxy terminus of an oligopeptide.

In a further embodiment, the present invention relates to a compound of formula I and the attendant definitions, wherein Y is NH and X is —CO-alkyl or the carboxy terminus of an N-acyl amino acid or the carboxy terminus of an oligopeptide.

In a further embodiment, the present invention relates to a compound of formula I and the attendant definitions, wherein D is a radical of oxycodone, W is —CH₂CH₂CH₂— or —CH₂CH₂CH₂CH₂—, and Y is NH.

In a further embodiment, the present invention relates to a compound of formula I and the attendant definitions, wherein D is a radical of oxycodone, W is —CH₂CH₂CH₂— or —CH₂CH₂CH₂CH₂—, and X is —CO-alkyl or the carboxy terminus of an N-acyl amino acid or the carboxy terminus of an oligopeptide.

In a further embodiment, the present invention relates to a compound of formula I and the attendant definitions, wherein D is a radical of oxycodone, Y is NH, and X is —CO-alkyl or the carboxy terminus of an N-acyl amino acid or the carboxy terminus of an oligopeptide.

In a further embodiment, the present invention relates to a compound of formula I and the attendant definitions, wherein W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

In a further embodiment, the present invention relates to a compound of formula I and the attendant definitions, wherein D is a radical of oxycodone, W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

Also included in the compounds of the present invention are pharmaceutically acceptable addition salts and complexes of the compounds of formula I. In cases wherein the compounds may have one or more chiral centers, unless specified, the present invention comprises each unique racemic compound, as well as each unique nonracemic compound.

In cases in which the compounds have unsaturated carbon-carbon double bonds, both the cis (Z) and trans (E) isomers are within the scope of this invention. In cases wherein compounds may exist in tautomeric forms, such as keto-enol tautomers, such as

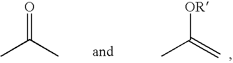

each tautomeric form is contemplated as being included within this invention, whether existing in equilibrium or locked in one form by appropriate substitution with R′. The meaning of any substituent at any one occurrence is independent of its meaning, or any other substituent's meaning, at any other occurrence.

Drugs

There are many drugs that it is desirable to deliver using the formulation described herein. The Controlled Substances Act (CSA), Title II of the Comprehensive Drug Abuse Prevention and Control Act of 1970, places all substances that are regulated under existing federal law into one of five schedules based upon the substance's medicinal value, harmfulness, and potential for abuse or addiction. Drugs include those classified as schedule II, III and IV drugs. Other drugs include those, like oxycodone, that are currently formulated as delayed or controlled release compositions, where drug release is intended to occur over a prolonged period of time through the gastrointestinal tract, and immediate or burst release, for example, by inhalation or injection, is undesirable. The drugs used in the formulation described herein possess a nucleophilic chemical group capable of reacting with an active carbonyl group to form a prodrug.

Examples of drugs include morphine and related compounds including alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, and tramadol.

In addition to the morphine-related compounds above, the following scheduled drugs may be incorporated into the formulation described in this invention in order to encourage proper, sustained release administration via the gastrointestinal tract: allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, and zolazepam hydrochloride.

Synthesis of Compounds

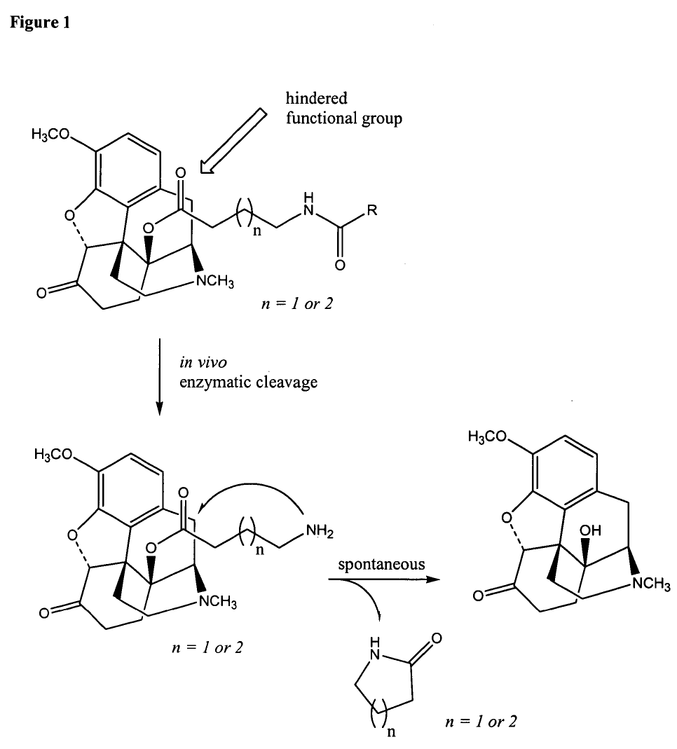

This approach utilizes a relay to convert a prodrug into the active substance. The relay may liberate the active drug by a two-step process. In the first step, enzymatic cleavage (in the gut) of the distal group (see FIG. 1) creates a free nucleophile. In the second step, the nucleophile, formed in step one, attacks the carbonyl group of the ester that is connected to, in the case of oxycodone, what was the OH group of oxycodone, effectively cleaving the ester and releasing oxycodone.

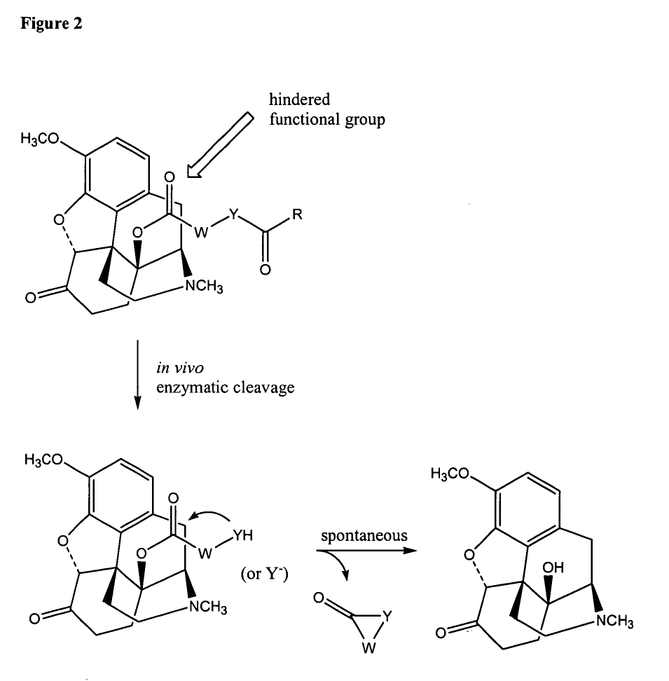

There may be various R groups allowing for the tuning of the release rate. In one embodiment, the “sidechain” is chemically innocuous, to the extent possible. The sidechain or linker may be a variety of chain lengths, compositions, as shown in FIG. 2. The linker may also be selected to increase the difficulty of degradation or increased resistance to enzyme or simple chemical hydrolysis.

Mixtures of linkages may also be used, for example, short and long linkages may be used to couple the R group to the drug.

It will also be recognized by one of ordinary skill in the art that the two groups on either end of the organic chain, i.e., the group that bonds to the drug and the group that undergoes enzymatic cleavage, will allow for tuning the release rate. For example, it is art recognized that the delocalization of the lone pair of electrons on the nitrogen over the adjacent carbonyl group generally makes amides more stable than esters. It is envisioned, therefore, that amides as the distal group will possess the required properties of being resistant to ordinary hydrolysis but susceptible to enzymatic cleavage. Enzymatic cleavage of an amide leaves behind an amine nucleophile which reacts intramolecularly to liberate the active form of the drug. If the group attached to the drug is an ester, then the overall reaction is thermodynamically favored with formation of an amide being the driving force behind the liberation of the drug.

Methods for manufacture and systhesis of the drug matrices and prodrugs described above are known to those skilled in the art, and the necessary reagents are commercially available.

Methods of the Present Invention

In part, the present invention relates to method of treating a mammal for pain relief comprising orally administering to the mammal a compound of formula I:

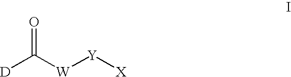

wherein:

D is a drug radical of a pain relief drug;

W is an organic chain comprising 3-5 carbon atoms that are substituted or unsubstituted and optionally comprises 3-5 heavy atoms selected from the group consisting of O, S, N, Si, and P;

Y is NH, S, or O; and

X is —CO-alkyl, —CO-aryl, —CO-aralkyl, —CO-heteroaryl, —CO-heteroaralkyl, —CO₂-alkyl, —CO₂-aryl, —CO-NHalkyl, —CO-NHaryl, the carboxy terminus of an N-acyl amino acid or the carboxy terminus of an oligopeptide.

In a further embodiment, the present invention relates to a method of treating a mammal for pain relief comprising administering to the mammal a compound of formula I and the attendant definitions, wherein D is a radical of alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, tramadol, allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, or zolazepam hydrochloride.

In a further embodiment, the present invention relates to a method of treating a mammal for pain relief comprising orally administering to the mammal a compound of formula I and the attendant definitions, wherein D is a radical of oxycodone.

In a further embodiment, the present invention relates to a method of treating a mammal for pain relief comprising orally administering to the mammal a compound of formula I and the attendant definitions, wherein W is —CH₂CH₂CH₂— or —CH₂CH₂CH₂CH₂—.

In a further embodiment, the present invention relates to a method of treating a mammal for pain relief comprising orally administering to the mammal a compound of formula I and the attendant definitions, wherein Y is NH.

In a further embodiment, the present invention relates to a method of treating a mammal for pain relief comprising orally administering to the mammal a compound of formula I and the attendant definitions, wherein X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein D is a radical of oxycodone and W is —CH₂CH₂CH₂— or —CH₂CH₂CH₂CH₂—.

In a further embodiment, the present invention relates to a method of treating a mammal for pain relief comprising orally administering to the mammal a compound of formula I and the attendant definitions, wherein D is a radical of oxycodone and Y is NH.

In a further embodiment, the present invention relates to a method of treating a mammal for pain relief comprising orally administering to the mammal a compound of formula I and the attendant definitions, wherein D is a radical of oxycodone and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein W is —CH₂CH₂CH₂— or —CH₂CH₂CH₂CH₂—; and Y is NH.

In a further embodiment, the present invention relates to a method of treating a mammal for pain relief comprising orally administering to the mammal a compound of formula I and the attendant definitions, wherein W is —CH₂CH₂CH₂— or —CH₂CH₂CH₂CH₂—; and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein Y is NH and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein D is a radical of oxycodone, W is —CH₂CH₂CH₂— or —CH₂CH₂CH₂CH₂—, and Y is NH.

In a further embodiment, the present invention relates to a method of treating a mammal for pain relief comprising orally administering to the mammal a compound of formula I and the attendant definitions, wherein D is a radical of oxycodone, W is —CH₂CH₂CH₂— or —CH₂CH₂CH₂CH₂—, and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein D is a radical of oxycodone, Y is NH, and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein D is a radical of oxycodone, W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

In a further embodiment, the present invention relates to a method of treating a mammal for pain relief comprising orally administering to the mammal a compound of formula I and the attendant definitions, wherein the mammal is a primate, equine, canine or feline. In a further embodiment, the mammal is a human.

In part, the present invention relates to a method of making a drug more abuse resistant comprising bonding a radical of formula II to the drug via a nucleophile present on the drug.

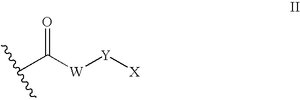

wherein:

W is an organic chain comprising 3-5 carbon atoms that are substituted or unsubstituted and optionally comprises 3-5 heavy atoms selected from the group consisting of O, S, N, Si, and P;

Y is NH, S, or O; and

X is —CO-alkyl, —CO-aryl, —CO-aralkyl, —CO-heteroaryl, —CO-heteroaralkyl, —CO₂-alkyl, —CO₂-aryl, —CO-NHalkyl, —CO-NHaryl, the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein the drug is alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, tramadol, allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, or zolazepam hydrochloride.

In a further embodiment, the present invention relates to a method of making a drug more abuse resistant comprising bonding a radical of formula II and the attendant definitions to the drug through a nucleophile present on the drug, wherein the drug is oxycodone.

In a further embodiment, the present invention relates to a method of making a drug more abuse resistant comprising bonding a radical of formula II and the attendant definitions to the drug through a nucleophile present on the drug, wherein W is —CH₂CH₂CH₂— or —CH₂CH₂CH₂CH₂—.

In a further embodiment, the present invention relates to a method of making a drug more abuse resistant comprising bonding a radical of formula II and the attendant definitions to the drug through a nucleophile present on the drug, wherein Y is NH.

In a further embodiment, the present invention relates to a method of making a drug more abuse resistant comprising bonding a radical of formula II and the attendant definitions to the drug through a nucleophile present on the drug, wherein X is —CO-alkyl or the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein the drug is oxycodone and W is —CH₂CH₂CH₂— or —CH₂CH₂CH₂CH₂—.

In a further embodiment, the present invention relates to a method of making a drug more abuse resistant comprising bonding a radical of formula II and the attendant definitions to the drug through a nucleophile present on the drug, wherein the drug is oxycodone and Y is NH.

In a further embodiment, the present invention relates to a method of making a drug more abuse resistant comprising bonding a radical of formula II and the attendant definitions to the drug through a nucleophile present on the drug, wherein the drug is oxycodone and X is —CO-alkyl or the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein Y is NH and X is —CO-alkyl or the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein the drug is oxycodone, W is —CH₂CH₂CH₂— or —CH₂CH₂CH₂CH₂—, and Y is NH.

In a further embodiment, the present invention relates to a method of making a drug more abuse resistant comprising bonding a radical of formula II and the attendant definitions to the drug through a nucleophile present on the drug, wherein the drug is oxycodone, W is —CH₂CH₂CH₂— or —CH₂CH₂CH₂CH₂—, and X is —CO-alkyl or the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein the drug is oxycodone, Y is NH, and X is —CO-alkyl or the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

In a further embodiment, the present invention relates to a method of making a drug more abuse resistant comprising bonding a radical of formula II and the attendant definitions to the drug through a nucleophile present on the drug, wherein the drug is oxycodone, W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

Gastrointestinal Enzymes

Gastrointestinal enzymes include the enzymes commonly known in the art to assist in the breaking down of food in the gastrointestinal system. The gastrointestinal system includes the stomach and related organs, including the pancreas, small intestine, and large intestine (colon). These enzymes aide digestion by hydrolyzing, for example, peptide bonds, starches, nucleic acids, and disaccharides. The enzymes in the gastrointestinal tract are capable of hydrolyzing the distal group of the prodrug at a greater rate than the carbonyl containing functional group that is connected to the nucleophile of the active drug. By this mechanism the active drug remains substantially as the inactive prodrug until liberated by the gastrointestinal enzymes.

Non-limiting examples of gastrointestinal enzymes include the proteolytic enzyme pepsin which cleaves peptide bonds, favoring those on the C-terminal side of tyrosine, phenylalanine, and tryptophan residues. Its role of breaking long polypeptide chains into shorter lengths allows it to hydrolize amide containing distal groups. Other gastrointestinal enzymes include pancreatic amylase which hyrdrolyzes starch into a mixture of maltose and glucose; pacreatic lipase which hydrolyzes ingested fats into a mixture of fatty acids and monglycerides; trypsin which cleaves peptide bonds on the C-terminal side of arginines and lysines; chymotrypsin which cleaves the C-terminal side of tyrosine, phenylalanine, and tryptophan residues; elastase which cuts peptide bonds next to small, uncharged side chains such as those of alanine and serine; carboxypeptidases which removes, one by one, the amino acids at the C-terminal of peptides; nucleases which hydrolyze ingested nucleic acids (RNA and DNA) into their component nucleotides; aminopeptidases which attack the amino terminal (N-terminal) of peptides producing amino acids; and disaccharidases which convert disaccharides into their monosaccharide subunits and include maltase which hydrolyzes maltose into glucose, sucrase which hydrolyzessucrose into glucose and fructose, and lactase which hydrolyzes lactose into glucose and galactose. Other non-limiting examples include pancreatin of multiple strength, pancrelipase, chymotrypsin B, pacreatopeptidase, carboxypeptidase A, carboxypeptidase B, glycerol ester hydrolase, ribonuclease, deoxyribonuclease, α-amylase, papain, chymopapain, bromelain, ficin, β-amylase, and cellulase.

Dosages

The dosage of any compositions of the present invention will vary depending on the symptoms, age and body weight of the patient, the nature and severity of the disorder to be treated or prevented, the route of administration, and the form of the subject composition. Any of the subject formulations may be administered in a single dose or in divided doses. Dosages for the compositions of the present invention may be readily determined by techniques known to those of skill in the art or as taught herein.

In certain embodiments, the dosage of the subject compounds will generally be in the range of about 0.01 ng to about 10 g per kg body weight, specifically in the range of about 1 ng to about 0.1 g per kg, and more specifically in the range of about 100 ng to about 10 mg per kg.

An effective dose or amount, and any possible affects on the timing of administration of the formulation, may need to be identified for any particular composition of the present invention. This may be accomplished by routine experiment as described herein, using one or more groups of animals (preferably at least 5 animals per group), or in human trials if appropriate. The effectiveness of any subject composition and method of treatment or prevention may be assessed by administering the composition and assessing the effect of the administration by measuring one or more applicable indices, and comparing the post-treatment values of these indices to the values of the same indices prior to treatment.

The precise time of administration and amount of any particular subject composition that will yield the most effective treatment in a given patient will depend upon the activity, pharmacokinetics, and bioavailability of a subject composition, physiological condition of the patient (including age, sex, disease type and stage, general physical condition, responsiveness to a given dosage and type of medication), route of administration, and the like. The guidelines presented herein may be used to optimize the treatment, e.g., determining the optimum time and/or amount of administration, which will require no more than routine experimentation consisting of monitoring the subject and adjusting the dosage and/or timing.

While the subject is being treated, the health of the patient may be monitored by measuring one or more of the relevant indices at predetermined times during the treatment period. Treatment, including composition, amounts, times of administration and formulation, may be optimized according to the results of such monitoring. The patient may be periodically reevaluated to determine the extent of improvement by measuring the same parameters. Adjustments to the amount(s) of subject composition administered and possibly to the time of administration may be made based on these reevaluations.

Treatment may be initiated with smaller dosages which are less than the optimum dose of the compound. Thereafter, the dosage may be increased by small increments until the optimum therapeutic effect is attained.

The use of the subject compositions may reduce the required dosage for any individual agent contained in the compositions because the onset and duration of effect of the different agents may be complimentary.

Toxicity and therapeutic efficacy of subject compositions may be determined by standard pharmaceutical procedures in cell cultures or experimental animals, e.g., for determining the LD₅₀ and the ED₅₀.

The data obtained from the cell culture assays and animal studies may be used in formulating a range of dosage for use in humans. The dosage of any subject composition lies preferably within a range of circulating concentrations that include the ED₅₀ with little or no toxicity. The dosage may vary within this range depending upon the dosage form employed and the route of administration utilized. For compositions of the present invention, the therapeutically effective dose may be estimated initially from cell culture assays.

Formulation

The compositions of the present invention may be administered orally. They may, for example, be formulated as tablets, capsules, granules, powders or syrups. These formulations may be prepared by conventional means, and, if desired, the compositions may be mixed with any conventional additive, such as an excipient, a binder, a disintegrating agent, a lubricant, a corrigent, a solubilizing agent, a suspension aid, an emulsifying agent or a coating agent.

In formulations of the subject invention, wetting agents, emulsifiers and lubricants, such as sodium lauryl sulfate and magnesium stearate, as well as coloring agents, release agents, coating agents, sweetening, flavoring and perfuming agents, preservatives and antioxidants may be present in the formulated agents.

The formulations may conveniently be presented in unit dosage form and may be prepared by any methods well known in the art of pharmacy. The amount of composition that may be combined with a carrier material to produce a single dose vary depending upon the subject being treated, and the particular mode of administration.

Methods of preparing these formulations include the step of bringing into association compositions of the present invention with the carrier and, optionally, one or more accessory ingredients. In general, the formulations are prepared by uniformly and intimately bringing into association agents with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present invention may also be administered as a bolus, electuary, or paste.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Kits

This invention also provides kits for conveniently and effectively implementing the methods of this invention. Such kits comprise any subject composition, and a means for facilitating compliance with methods of this invention. Such kits provide a convenient and effective means for assuring that the subject to be treated takes the appropriate active in the correct dosage in the correct manner. The compliance means of such kits includes any means which facilitates administering the actives according to a method of this invention. Such compliance means include instructions, packaging, and dispensing means, and combinations thereof. Kit components may be packaged for either manual or partially or wholly automated practice of the foregoing methods. In other embodiments involving kits, this invention contemplates a kit including compositions of the present invention, and optionally instructions for their use.

### Incorporation by Reference

All of the patents and publications cited herein are hereby incorporated by reference.

### Equivalents

Those skilled in the art will recognize, or be able to ascertain using no more than routine experimentation, many equivalents to the specific embodiments of the invention described herein. Such equivalents are intended to be encompassed by the following claims.

We claim:

1. A compound comprising a drug attached to an organic chain via a nucleophile present on the drug, wherein the organic chain has, at the end distal to the drug, an organic group susceptible to enzymatic cleavage and substantially unsusceptible to non-enzymatic cleavage.

2. The compound of claim 1, wherein the distal organic group is an amide.

3. The compound of claim 1, wherein the nucleophile and the organic chain form an ester.

4. The compound of claim 3, wherein the ester is cleaved via an intramolecular reaction after enzymatic cleavage of the distal organic group.

5. The compound of claim 4, wherein the distal organic group is an amide.

6. The compound of claim 1, wherein the drug is a pain relief drug.

7. The compound of claim 1, wherein the drug is alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, tramadol, allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, or zolazepam hydrochloride.

8. The compound of claim 1, wherein the drug is oxycodone.

9. A pharmaceutical composition, comprising the compound of claim 1; and a pharmaceutically acceptable excipient.

10. A method of treating pain in a mammal, comprising administering to a mammal in need thereof a therapeutically effective amount of the compound of claim 6.

11. The method of claim 10, wherein the mammal is a primate, equine, canine, or feline.

12. The method of claim 10, wherein the mammal is a human.

13. A method of making a drug more difficult to abuse, comprising bonding an organic radical to the drug at one end of the organic radical via a nucleophile on the drug, wherein said organic radical comprises a functional group that is susceptible to enzymatic cleavage and substantially unsusceptible to non-enzymatic cleavage.

14. A kit comprising the compound of claim 1 and instructions for use thereof.

15. A compound of formula I: 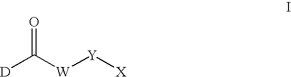
wherein
D is a drug radical;
W is an organic chain comprising 3-5 carbon atoms that are substituted or unsubstituted and optionally comprises 3-5 heavy atoms selected from the group consisting of 0. S, N, Si, and P;
Y is NH, S, or O; and
X is —CO-alkyl, —CO-aryl, —CO-aralkyl, —CO-heteroaryl, —CO-heteroaralkyl, —CO₂-alkyl, —CO₂-aryl, —CO-NHalkyl, —CO-NHaryl, the carboxy terminus of an N-acyl amino acid or the carboxy terminus of an oligopeptide.

16. The compound of claim 15, wherein D is a radical of a pain relief drug.

17. The compound of claim 15, wherein D is a radical of alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, tramadol, allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, or zolazepam hydrochloride.

18. The compound of claim 15, wherein D is a radical of oxycodone.

19. The compound of claim 15, wherein W is —CH₂CH₂CH₂— or —CH₂CH₂CH₂CH₂—.

20. The compound of claim 15, wherein Y is NH.

21. The compound of claim 15, wherein X is —CO-alkyl or the carboxy terminus of an N-acyl amino acid or the carboxy terminus of an oligopeptide.

22. The compound of claim 15, wherein D is a radical of oxycodone and W is —CH₂CH₂CH₂ —or —CH₂CH₂CH₂CH₂—.

23. The compound of claim 15, wherein D is a radical of oxycodone and Y is NH.

24. The compound of claim 15, wherein D is a radical of oxycodone and X is —CO-alkyl or the carboxy terminus of an N-acyl amino acid or the carboxy terminus of an oligopeptide.

25. The compound of claim 15, wherein W is —CH₂CH₂CH₂— or —CH₂CH₂CH₂CH₂—; and Y is NH.

26. The compound of claim 15, wherein W is —CH₂CH₂CH₂— or —CH₂CH₂CH₂CH₂—; and X is —CO-alkyl or the carboxy terminus of an N-acyl amino acid or the carboxy terminus of an oligopeptide.

27. The compound of claim 15, wherein Y is NH and X is —CO-alkyl or the carboxy terminus of an N-acyl amino acid or the carboxy terminus of an oligopeptide.

28. The compound of claim 15, wherein D is a radical of oxycodone, W is —CH₂CH₂CH₂— or —CH₂CH₂CH₂CH₂—, and Y is NH.

29. The compound of claim 15, wherein D is a radical of oxycodone, W is —CH₂CH₂CH₂— or —CH₂CH₂CH₂CH₂—, and X is —CO-alkyl or the carboxy terminus of an N-acyl amino acid or the carboxy terminus of an oligopeptide.

30. The compound of claim 15, wherein D is a radical of oxycodone, Y is NH, and X is —CO-alkyl or the carboxy terminus of an N-acyl amino acid or the carboxy terminus of an oligopeptide.

31. The compound of claim 15, wherein W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

32. The compound of claim 15, wherein D is a radical of oxycodone, W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

33. A pharmaceutical composition, comprising the compound of claim 15; and a pharmaceutically acceptable excipient.

34. A kit comprising the compound of claim 15 and instructions for use thereof.

35. A method of treating pain in a mammal, comprising administering to a mammal in need thereof a therapeutically effective amount of the compound of claim 16.

36. The method of claim 35, wherein the mammal is a primate, equine, canine or feline.

37. The method of claim 35, wherein the mammal is a human.

38. A method of making a drug more difficult to abuse, comprising bonding to a nucleophile on the drug a radical of formula II: 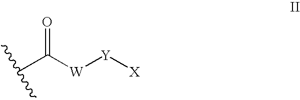
wherein
W is an organic chain comprising 3-5 carbon atoms that are substituted or unsubstituted and optionally comprises 3-5 heavy atoms selected from the group consisting of O, S, N, Si, and P;
Y is NH, S, or O; and
X is —CO-alkyl, —CO-aryl, —CO-aralkyl, —CO-heteroaryl, —CO-heteroaralkyl, —CO₂-alkyl, —CO₂-aryl, —CO-NHalkyl, —CO-NHaryl, the carboxy terminus of an N-acyl amino acid or the carboxy terminus of an oligopeptide.

39. The method of claim 38, wherein the drug is a pain relief drug.

40. The method of claim 38, wherein the drug is alphacetylmethadol hydrochloride, anileridine, apomorphine, bemidone, betacetylmethadol hydrochloride, buprenorphine hydrochloride, butorphanol tartrate, codeine, dezocine, dihydrocodeine, dihydromorphine, dipanone hydrochloride, eptazocine hydrobromide, ethylmorphine, etorphine hydrochloride, hydromorphone, ketobemidone, levorphanol tartrate, loperamide, meptazinol hydrochloride, methyldihydromorphinone, nalbuphine hydrochloride, nalbuphine hydrochloride, normorphine, oxycodone, oxymorphone, pentazocine, piminodine, tramadol, allobarbitone, alprazolan, amylobarbitone, barbitone sodium, butobarbitone, captodiame hydrochloride, chloral betaine, chloral hydrate, chloralose, chlorhexadol, chlormethiazole edisylate, cinolazepam, potassium clorazepate, cyclobarbitone calcium, delorzepam, difebarbamate, enciprazine hydrochloride, flunitrazepam, hexobarbitone sodium, ibomal, lorazepam, lormetazepam, meprobamate, methylpentynol, midazolam maleate, oxazepam, pentabarbitone calcium, phenprobamate, proxibarbal, quinalbaritone, quinalbarbitone sodium, secbutobarbitone sodium, temazepam, triclofos sodium, zalepan, or zolazepam hydrochloride.

41. The method of claim 38, wherein the drug is oxycodone.

42. The method of claim 38, wherein W is —CH₂CH₂CH₂— or —CH₂CH₂CH₂CH₂—.

43. The method of claim 38, wherein Y is NH.

44. The method of claim 38, wherein X is —CO-alkyl or the carboxy terminus of an N-acyl amino acid or the carboxy terminus of an oligopeptide.

45. The method of claim 38, wherein the drug is oxycodone; and W is W is —CH₂CH₂CH₂— or —CH₂CH₂CH₂CH₂—.

46. The method of claim 38, wherein the drug is oxycodone; and Y is NH.

47. The method of claim 38, wherein the drug is oxycodone; and X is —CO-alkyl or the carboxy terminus of an N-acyl amino acid or the carboxy terminus of an oligopeptide.

48. The method of claim 38, wherein Y is NH; and X is —CO-alkyl or the carboxy terminus of an N-acyl amino acid or the carboxy terminus of an oligopeptide.

49. The method of claim 38, wherein the drug is oxycodone, W is —CH₂CH₂CH₂— or —CH₂CH₂CH₂CH₂—, and Y is NH.

50. The method of claim 38, wherein the drug is oxycodone, W is —CH₂CH₂CH₂— or —CH₂CH₂CH₂CH₂—, and X is —CO-alkyl or the carboxy terminus of an N-acyl amino acid or the carboxy terminus of an oligopeptide.

51. The method of claim 38, wherein the drug is oxycodone, Y is NH, and X is —CO-alkyl or the carboxy terminus of an N-acyl amino acid or the carboxy terminus of an oligopeptide.

52. The method of claim 38, wherein W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.

53. The method of claim 38, wherein the drug is oxycodone, W is —CH₂CH₂CH₂— or —CH₂CH₂CH₂CH₂—, Y is NH, and X is —CO-alkyl or the carboxy terminus of an N-acyl amino acid or the carboxy terminus of an oligopeptide.
